(19) United States
(12) Patent Application Publication
(10) Pub. No.: US 2019/0060106 A1
(43) Pub. Date: Feb. 28, 2019

(54) Zone-Controlled Warming System

(71) Applicant: 3M INNOVATIVE PROPERTIES COMPANY, St. Paul, MN (US)

(72) Inventors: Matthew Zabel, Lake Elmo, MN (US); Michael Vostal, Minneapolis, MN (US); Poeuth Pann, Woodbury, MN (US); Jared J. Balthazor, Linwood TWP, MN (US); Melanie L. Collins, Minneapolis, MN (US); Philip G. Dion, Blaine, MN (US); Daniel P. Doran, Minneapolis, MN (US); Marc A. Egeland, Minneapolis, MN (US); Yvonne Lund, Roberts, WI (US); Glenn R. Maharaj, Minneapolis, MN (US); Andrew J. McGregor, West Lakeland, MN (US); John R. Stark, St. Paul, MN (US)

(21) Appl. No.: 16/109,247

(22) Filed: Aug. 22, 2018

Related U.S. Application Data
(60) Provisional application No. 62/549,224, filed on Aug. 23, 2017.
(60) Provisional application No. 62/549,302, filed on Aug. 23, 2017.

(51) Int. Cl. A61F 7/00; A61B 5/01; A61B 5/00

(52) U.S. Cl. CPC A61F 7/007; H05B 3/36; A61B 5/4836; A61B 5/01

(57) ABSTRACT

Aspects of the present disclose related to a system. The system includes a warming device having at least a first zone and a second zone and a sensor. The system also includes a controller that includes one or more processor circuits configured to receive a first sensor reading from the sensor corresponding to a first zone, wherein the first sensor reading corresponds to a first heat transfer rate. The one or more processor circuits are configured to determine whether the sensor reading is sufficient and increase a first heat transfer rate to a second heat transfer rate in the first zone in response to the sensor reading being insufficient.

### BACKGROUND

Warming a person during surgery affords clinical benefits, such as prevention or treatment of hypothermia, encouragement of immune system function, promotion of wound healing, reduction of infection rates, and mitigation of discomfort.

Various designs of convective and conductive warming devices have been proposed. However, the warming devices generally are limited by transfer of heat to an overall area and not on specific areas of heating. Further, warming devices generally rely on electrical power of a building which can limit mobility of an overall warming system.

### SUMMARY

Aspects of the present disclosure are related to a system. The system includes a warming device having at least a first zone and a second zone and a sensor. The system also includes a controller that includes one or more processor circuits configured to receive a first sensor reading from the sensor corresponding to a first zone, wherein the first sensor reading corresponds to a first heat transfer rate. The one or more processor circuits are configured to determine whether the sensor reading is sufficient and increase a first heat transfer rate to a second heat transfer rate in the first zone in response to the sensor reading being insufficient.

### BRIEF DESCRIPTION OF DRAWINGS

FIG. 1 illustrates a block diagram of a zone controlled warming system utilizing one or more sensors and control valves, according to aspects of the present disclosure.

FIG. 2 illustrates a block diagram of one or more sensors operable with the zone-controlled warming system, according to aspects of the present disclosure.

FIG. 3 illustrates a flowchart of a method of determining whether to transfer additional heat to or from a zone of a warming device, according to aspects of the present disclosure.

FIG. 4 illustrates a flowchart of a method of determining the transfer of additional heat to the zone of a warming device, according to aspects of the present disclosure.

FIG. 5 illustrates a flowchart of a method of determining heating settings for a transfer of heat from a heat source, according to aspects of the present disclosure.

FIG. 6 illustrates a top elevational view of an embodiment of a convective zone-controlled warming system, according to aspects of the present disclosure.

FIG. 7A illustrates a hose system of the embodiment in FIG. 6, according to aspects of the present disclosure.

FIG. 7B illustrates a front view of the embodiment in FIG. 7A taken along lines 1-1, according to aspects of the present disclosure.

FIG. 8 illustrates a top elevational view of an embodiment of a convective warming device, according to aspects of the present disclosure.

FIG. 9A illustrates a top elevational view of an embodiment of a conductive zone-controlled warming system, according to aspects of the present disclosure.

FIG. 9B illustrates a side view of the embodiment in FIG. 9A taken along lines 2-2, according to aspects of the present disclosure.

### DETAILED DESCRIPTION

Aspects of the present disclosure relate to a warming device having multiple heating zones (“zones”) to heat a patient. The warming device can also have a heat transfer element to transfer heat from one zone to another zone thus reducing load on a heat source and conserving power. The warming device can also have a plurality of sensors to measure physiological parameters of the patient or physical parameters of a zone to inform a controller whether to increase a heat using the heat source or draw heat from another zone.

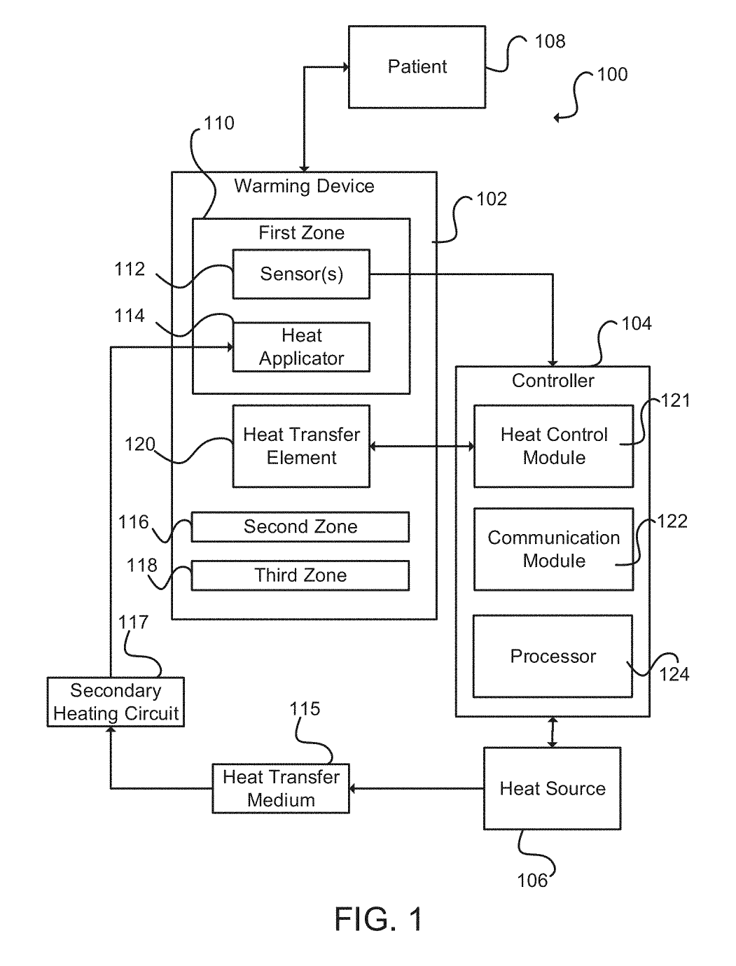

FIG. 1 illustrates a block diagram of a zone-controlled warming system 100 (“system”). The system 100 can include a warming device 102, a controller 104, a heat source 106, patient 108, and one or more sensors 112 disposed proximate to the warming device 102.

The patient 108 can be thermally coupled to a portion of the warming device 102. In at least one embodiment, the patient 108 is any mammalian animal, preferably a human. The patient 108 is capable of maintaining normothermia.

In at least one embodiment, the warming device 102 can be configured to provide heat from the heat source 106 to the patient 108. The warming device 102 can use a variety of form factors including a blanket, intravenous, gown, pad, bed, or combinations thereof. The warming device 102 can be configured to use a variety of different heat sources 106. For the purpose of conciseness, conductive heating or convective heating will be described in detail. Other heat transfer mechanisms are possible such as radiative, advection, or combinations thereof with conduction or convection. For example, advection heat transfer can be present in fluidic warming systems such as those sold by 3M under the trade designation Ranger. In another example, Infrared (IR) warming of the patient is also possible by using IR light within a bed or operating room table to warm the patient.

In at least one embodiment, the construction of the warming device 102 can use general principles that are known. For example, a conductive warming device 102 can use electrically conductive ink that provides a resistive element to an electrical current sufficient for the resistive element to generate heat such as those described in WO2016186671 by Steffan. The construction of a convective warming device 102 can be similar to the multi-layered construction as used in a warming blanket or gown such as those sold by 3M under the trade designation Bair Hugger.

The warming device 102 can have a plurality of zones (e.g., 110, 116, and 118). Generally, the zone refers to an area of heating. In at least one embodiment, a zone is thermally separated and does not overlap within another zone. For example, each zone may have different thermal characteristics from another zone.

The zones can be defined by a physical boundary, such as a border or formed through gaps where heat is not applied. In at least one embodiment, the zone can correspond to portions of the patient. For example, one zone can be aligned with the core of a patient and another zone can be aligned with the extremities (e.g., arms and legs) of the patient. The alignment of a zone with extremities can be particularly useful in a pre-warming procedure (i.e., warming before induction of anesthesia).

For the purpose of conciseness, the first zone 110 will be described in detail. The second zone 116, and third zone 118 may be of similar construction to the first zone 110. The first zone 110 can have a sensor 112 and a heat applicator 114.

The sensor 112 can be measuring device that measures one or more physiological parameters of the patient 108 or physical parameters of within first zone 110. The sensor 112 provides a plurality of sensor readings. In at least one embodiment, the sensor 112 can be configured to measure data sufficient to determine an actual heat transfer rate of the first zone 110. Due to measurement of the heat transfer rate, the sensor 112 may provide sensor readings to the controller 104 with sufficient regularity (for example, at least one per minute, at least once per 30 seconds, at least once per second). The sensor 112 can be placed in a location to optimize data collection. For example, using a convective heat source 106, an airflow sensor can be placed proximate to an inlet port (i.e., a port for receiving air), in a crevice, or combinations thereof. Similarly, a temperature sensor can be placed at a convective heat source 106, within a portion of the first zone 110, or combinations thereof. In addition, the sensor can be thermally separated (meaning that a negligible amount of heat from the first zone 110 is transferred to the sensor 112). For example, thermal separation can use a standoff distance between the heat applicator 114 and the sensor 112, insulation, or combinations thereof. Thus, the heat applicator 114 does not affect the sensor directly (although the heat applicator 114 can affect the sensor reading indirectly by transferring heat to the patient 108 and be measured). Various types of sensors may be described herein.

A heat applicator 114 can dissipate heat such that the heat from the heat source 106 is not concentrated (which may cause burning or irritation). For example, a heated coil, while efficient at energy transfer, will cause burns if applied directly to the patient's skin. The heat applicator 114 can have a construction or be formed of a material that enables distribution of heat across its surface and transfer to the patient 108 through the surface. For example, the heat applicator 114 can be a polymer, gel, thermally conductive metal, air, or liquid such as water. In a conductive system, the heat applicator 114 can be the resistive element or a material adjacent the resistive element that distributes the heat from the resistive element. In a convective system, the heat applicator 114 can be an air permeable layer where expelled warmed air is distributed and transferred to the patient 108. In a radiative system, the heat applicator 114 can be air that dissipates Infrared (IR) energy and/or a reflective surface. In at least one embodiment, the heat applicator 114 can also include a heat transfer medium 115 described herein.

The warming device 102 can also include a heat transfer element 120. The heat transfer element 120 is configured to transfer heat between proximate zones, preferably adjacent zones. The heat transfer element 120 is generally activatable, meaning that it can be electrically activated by the controller 104. In at least one embodiment, the heat transfer element 120 is a controllable valve (preferably an electromechanical valve) positioned between inflatable zones of a convective warming device 102. For example, each zone can be fluidically isolated from another zone except through the controllable valve.

In at least one embodiment, the heat transfer element 120 is a thermal mass that forms a thermal bridge between two zones. A thermal mass can be particularly useful in conductive systems. For example, a thermal bridge can be formed from high thermally conductive materials such as metals (e.g., copper and aluminum), and metal reinforced polymers. In another example, the thermal bridge can be formed by a liquid coolant which can transfer heat between zones with the aid of a controllable pumping mechanism. In such a system, each zone may further comprise a heat sink, tubing, and between each zone is a pump in a fluidically closed-loop system.

The system 100 can include a controller 104. The controller 104 includes a heat control module 121, a communication module 122, and one or more processor circuits 124. The controller 104 can have one or more modules such as a heat control module 121 that generally determine a heat transfer rate in a plurality of zones from the sensors 112. The heat control module 121 can determine when to activate the heat transfer element 120 based on a plurality of sensor readings.

The controller can also include a communication module 122. The communication module 122 can be configured to communicatively couple with sensors 112 and heat transfer element 120. The communication between various components can be wired or wireless. Due to patient 108 movement, the preferred communication is wireless. For example, the wireless communication from the communications module can use a Bluetooth protocol or Wi-Fi using IEEE 802.11 protocols or even ultra-wide band. Preferably, the wireless signal can operate using a medical body area network (MBAN) which can operate in the 2360-2390 MHz band or the 2390-2400 MHz band. In at least one embodiment, the communication is wireless between sensor 112 and heat transfer element 120 and the controller 104, but wired in other parts. The controller 104 can also include one or more processor circuits 124. The one or more processor circuits 124 can be a designed application specific integrated circuit (ASIC) which is communicatively coupled to the sensors 112 and the heat transfer element 120, and the heat source 106.

The system 100 can include a heat source 106. The heat source 106 provides thermal energy to the warming device 102. The heat source 106 can use a variety of heat transfer mechanisms as described herein.

In at least one embodiment, the heat source 106 is powered by a fixed power supply. The heat source 106 can be powered by a battery to allow portability of the system 100 throughout a hospital. Due to the energy required by a heat source 106, heat transfer between zones can be particularly advantageous to portable systems because of power savings as it is generally less energy intensive to operate heat transfer elements between zones rather than a convective heat source 106. An example of a convective heat source 106 can be the warming units commercially available under the trade designation Bair Hugger by 3M. In at least one embodiment, the convective heat source is a clinical warming unit and can have a heat transfer rate of between 1000 BTU/hr and 2000 BTU/hr (average), 1200 BTU/hr and 1800 BTU/hr (average), or preferably 1330 BTU/hr to 1600 BTU/hr (average). Similarly, in a conductive system, the heat source 106 can refer to the heating coil or resistive element. Likewise, in a radiative system, the heat source 106 can be a bulb.

The heat source 106 can thermally affect a heat transfer medium 115. The heat transfer medium is the medium that transfers heat from the heat source 106 to the heat applicator 114. In at least one embodiment, the heat transfer medium 115 is optional depending on the type of heat transfer mechanism. For example, the heat transfer medium 115 of a radiative system can have a medium of light. A heat transfer medium 115 of a conductive system is the same as or similar to the heat applicator 114 (e.g., a cover that distributes heat). A heat transfer medium 115 of a convective heat source 106 is air. Heat source 106 heats air, which is then routed through an air permeable layer (i.e., heat applicator 114) of the warming device 102, and warmed air (i.e., heat transfer medium 115) transfers energy onto the skin of the patient 108.

The system 100 can also have an optional secondary heating circuit 117. The secondary heating circuit 117 provides heat in addition to the heat source 106. In at least one embodiment, the secondary heating circuit 117 can comprise a resistive element coupled to a power source sufficient to increase the heat transfer rate of the heat source 106. In some embodiments, the secondary heating circuit 117 can be positioned downstream from the heat source 106. In at least one embodiment, the secondary heating circuit 117 can be a comfort warming unit, such as those commercially available under the trade designation Bair Hugger under model number 875 from 3M. In at least one embodiment, a comfort warming unit (e.g., a secondary heating circuit) can be used to increase the thermal performance of another comfort warming unit (e.g., a heat source). In at least one embodiment, the comfort warming unit can have an output of no greater than 1200 BTU/hr and generally around 1000 BTU/hr.

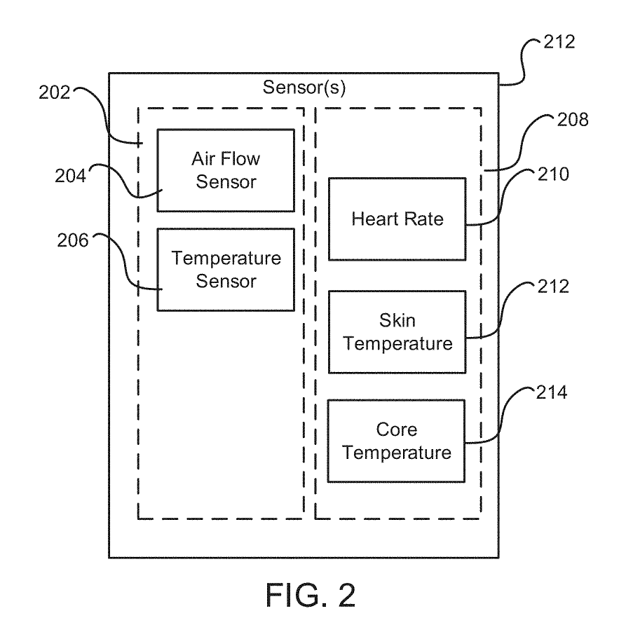

FIG. 2 illustrates one or more sensors 212. The sensors 212 can correspond to the sensors 112 from FIG. 1. The sensors 212 can generally be divided into those that measure physical parameters 202 of the warming device and those that measure physiological parameters 208.

Physical parameters 202 of the warming device can be measured for feedback of conditions within the warming device. Examples of sensors that measure physical parameters 202 include an air flow sensor 204, and a temperature sensor 206.

The air flow sensor 204 senses the flow of air within a zone (preferably in a convective warming device). The air flow sensor 204 can be useful in determining the heat transfer rate to a patient. For example, higher airflow can result in more heat transfer at the same temperature. Alternatively, if the air temperature is lower than body temperature, the higher airflow can cool a patient.

A temperature sensor 206 generally records temperature. Temperature sensors can be used that are portable and preferably a flat format. Various temperature sensors 206 can be used such as resistance temperature detector, thermocouples, thermistors, contact, and remote temperature sensors.

Physiological parameters 208 of the patient can also be measured. However, some parameters 208, like core body temperature may vary little between zones. Thus, a non-localized sensor measuring physiological parameters 208 may be used in conjunction with at least a sensor localized to the zone.

Examples of sensors that measure physiological parameters 208 include heart rate 210, skin temperature 212, and core temperature 214. For example, pulse can be connected to vasodilation of a patient and is indicative of when to reduce heat of the warming device. A core temperature sensor 214 can measure the core temperature of a patient. Various core temperature sensors exist such as ingestible sensors and a zero-heat flux thermometer such as those commercially available under the trade designation SpotOn® by 3M.

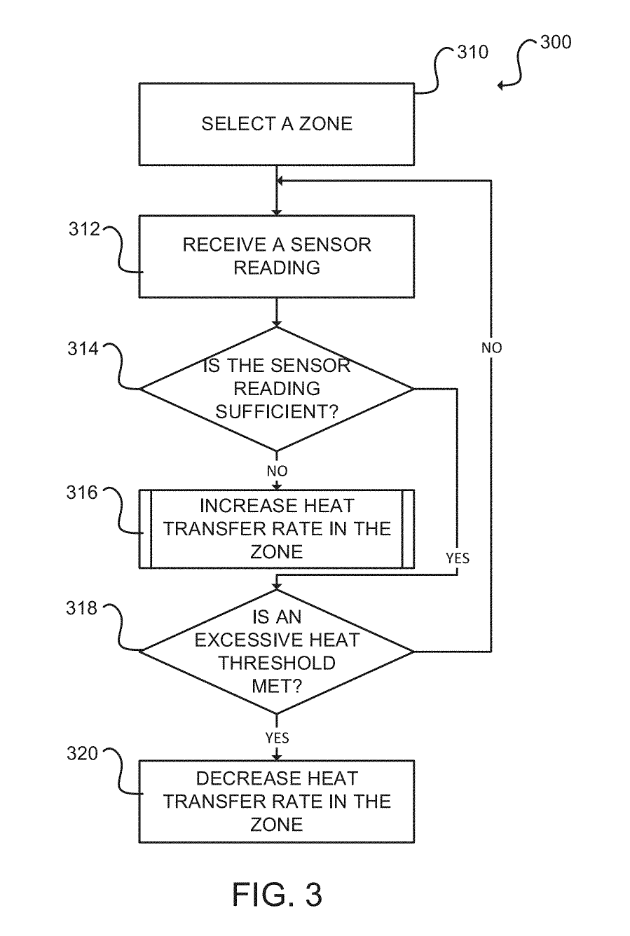

FIG. 3 illustrates a flowchart of a method 300 for determining whether to increase or decrease the heat transfer rate within a zone. Generally, heat may transfer from the heat source, through the zone of the warming device, and to a portion of the patient corresponding to the zone at a heat transfer rate. The transfer of additional heat as used herein refers to increasing the heat transfer rate from a first transfer rate to a second transfer rate. Since the first transfer rate can be negligible between zones (unless the heat transfer element has previously facilitated transfer), then the second transfer rate can be non-zero. In at least one embodiment, the decreasing of the heat transfer rate in the zone can be removing heat from the zone since a total amount of heat transferred is lower.

The method can begin at block 310. In block 310, the controller can select a zone. In at least one embodiment, the zone can be an area that is defined by the heat applicator and/or the thermal characteristics of the warming device. For example, in a convective warming device, the shape of a chamber can form the boundaries of the zone. In a conductive warming device, the shape of the resistive element can define the boundaries of the zone.

In block 312, the controller can receive a first sensor reading from a sensor corresponding to a first zone. The sensor is generally located within the zone (however, a core temperature sensor is unlikely to be located within the zone). For example, a physiological sensor can communicate with the controller. The physiological sensor may be isolated to measurements within the zone. For example, a skin temperature sensor can measure the portion of the patient within the zone.

In block 314, the controller can determine whether the sensor reading is sufficient. In at least one embodiment, whether the sensor reading is sufficient can be based on a temperature.

For example, a sensor reading can be sufficient when the sensor reading indicates that the zone or the patient portion within the zone is at a range of temperatures. Generally, the range of temperatures is from 36 to 44 degrees C. Higher temperatures than normal body temperatures may also be possible where the patient is warmed faster at a higher temperature. For example, the temperature of at no greater than 44 degrees C. may be applied to an extremity of the patient to warm the core temperature of the patient. In at least one embodiment, the range of temperatures can correspond to the normal body temperature for the patient. For example, in a human adult, the range of temperatures can be from 36.3 to 37.3 degrees C. In at least one embodiment, the range of temperatures can also account for when the patient has undergone vasodilation and is about to sweat. In at least one embodiment, a sufficient sensor reading can at or above normothermia (e.g., 36 degrees C.). Thus, an insufficient sensor reading can be when the temperature is below 36 degrees C.

In at least one embodiment, an insufficient sensor reading can refer to an error in data collection for the sensor. For example, the sensor malfunctioned and is not able to be read or provides corrupted data.

In at least one embodiment, the sensor reading may indicate a heat transfer rate, i.e., the amount of heat provided to the patient per unit of time. For example, an airflow sensor and a temperature sensor can be used to estimate the actual heat transfer rate in the zone. Whether a sensor reading is sufficient can be based on whether a first heat transfer rate will cause the patient to maintain the range of temperatures, or the range of temperatures is present in the zone. In at least one embodiment, a first zone threshold can be defined by the likelihood of the patient maintaining the range of temperatures. The first zone threshold can further be based off of a physiological parameter like core body temperature. The controller can also determine whether the first heat transfer rate is within the first zone threshold to determine sufficient sensor readings.

In block 316, the controller can increase the heat transfer rate in the zone if the sensor reading is not sufficient. In at least one embodiment, the controller can increase the heat transfer rate by allowing heat to flow from another zone or by increasing the heat transfer rate from the heat source. For example, in a convective system, if the heat measured by the skin sensor is indicates a lower than normal body temperature for the patient, then the warming unit fan speed can increase to increase the heat delivered to the patient.

In block 318, the controller can determine whether an excessive heat threshold is met responsive to the sensor reading being insufficient. In at least one embodiment, the excessive heat threshold is a threshold that indicates when a zone temperature is above a heat transfer rate which may cause the patient to retain excessive heat. In at least one embodiment, the excessive heat threshold can be based on the normal body temperature for the region of the patient that the zone corresponds to. For example, for an extremity, the excessive heat threshold can be higher than normal body temperature. For example, the excessive heat threshold may be 44 degrees C. in the arms but 38 degrees C. in the core of a patient.

In at least one embodiment, the excessive heat threshold can correspond to temperatures of at least 44 degrees C. For example, temperatures of 49 degrees C. or higher are likely to cause burns, and temperatures of between 49 and 44 degrees C. may cause irritation or sweating. In at least one embodiment, the excessive heat threshold can be based on elevated temperatures (i.e., fever temperature) of a patient. For example, the excessive heat threshold may be no greater than 44, 43, 42, 41, or 40 degrees C.

In block 320, the controller can decrease the heat transfer rate in the zone in response to the excessive heat threshold being met. The controller can decrease the heat transfer rate in a variety of manners. For example, the controller can activate/open a heat transfer element to transfer a portion of heat from the first zone to a second zone. In at least one embodiment, depending on the degree of excessive heat, the controller can vent the heat into the atmosphere. The controller can also reduce the heat transfer rate of the heat source by deactivating a portion of the heat source or reducing the temperature or airflow level of the heat source.

If the excessive heat threshold is not met, then the controller can maintain heat settings of the heat source to maintain the heat transfer rate.

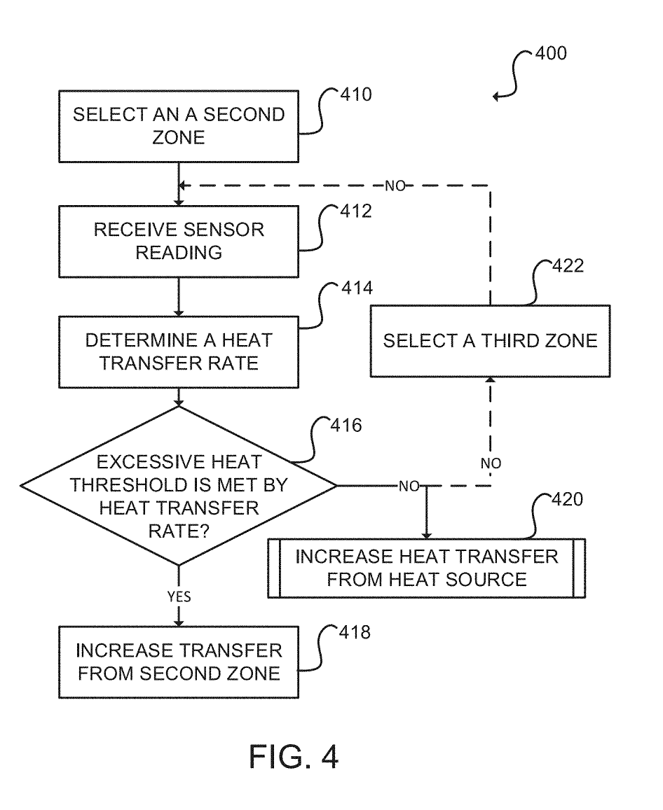

FIG. 4 illustrates a flowchart of a method 400 for increasing heat transfer rate in the zone. Method 400 can correspond to block 316 in FIG. 3. The method 400 can begin at block 410.

In block 410, the controller can select a second zone. The controller can select a second zone in response to the sensor reading not being sufficient in block 314 of method 300. In at least one embodiment, the second zone is an proximate zone that is not coupled (e.g., thermally) to the first zone. In some embodiments, the second zone is adjacent to the first zone.

In block 412, the controller can receive a sensor reading from a sensor within the second zone or corresponding to the second zone. For example, the sensor may measure a core body temperature which may correspond to a core of a patient. In at least one embodiment, the sensor from block 412 is the same type of sensor as in block 312. In at least one embodiment, the sensor can be measured from approximately the same location within the zone (e.g., measured within a crevice for both sensors or the same proximity to the heat source).

In block 414, the controller can determine a second zone heat transfer rate present in the second zone. The second zone heat transfer rate determination can be based on the sensor reading. A heat transfer rate refers to the rate of heat transfer from one object to another object. For example, from the heat applicator to the patient. The heat transfer rate can vary based on the interaction with the warming device. In at least one embodiment, the heat transfer rate of a convective warming device can be defined by air temperature, the airflow being applied to the patient, the specific heat of the material of the warming device, or combinations thereof. In at least one embodiment, the heat transfer rate of a conductive warming device can be defined by the temperature of the pad, any thermal masses or heat transfer media that retain heat, or combinations thereof.

In block 416, the controller can determine whether the excessive heat threshold is met by the second zone heat transfer rate. In at least one embodiment, the excessive heat threshold may be the same as determined in block 318. As discussed herein, the excessive heat threshold can be based on the portion of the patient corresponding to the zone. In at least one embodiment, if the heat transfer rate is excessive, then at least some of the heat can be transferred to the first zone or otherwise reduced for patient safety or comfort.

In block 418, the controller can increase a transfer of heat from the second zone responsive to the excessive heat threshold being met. For example, if the heat transfer element is closed/inactive, then the heat transfer rate between the first zone and the second zone is negligible. If activated in block 418, then the heat transfer element can increase the heat transfer rate from about zero to a second heat transfer rate between zones.

In block 420, the controller can increase the heat transfer rate in the first zone by increasing the heat transfer rate from the heat source in response to the excessive heat threshold not being met by the second zone.

In block 422, the controller can select a third zone. Thus, optionally, the controller can attempt to cure any deficiencies in the heat transfer rate in the first zone from any proximate zones before requesting further increases in the heat transfer rate from the heat source.

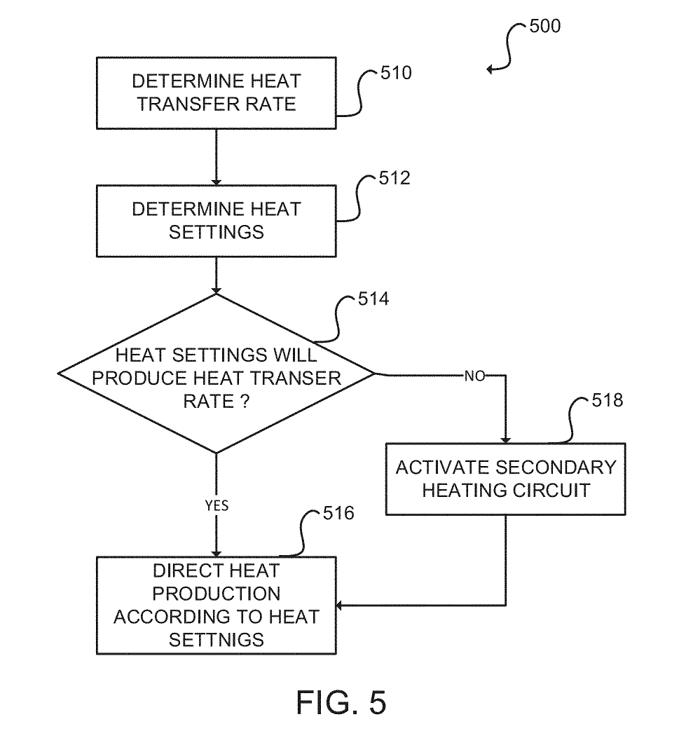

FIG. 5 illustrates a method 500 for increasing the heat transfer rate from the heat source. Method 500 can correspond to block 420 in FIG. 4.

In block 510, the controller can determine a heat transfer rate. In at least one embodiment, the heat transfer rate can be related to a current heat transfer rate of the heat source including any system losses. In at least one embodiment, a heat transfer rate can be a target heat transfer rate that can be determined based on how the heat transfer rate interacts, or is expected to interact with the patient at the zone. For example, if the first heat transfer rate is 3 calories per second, the current heat transfer rate of the heat source is 5 calories per second (i.e., loss of 2 calories to the system); the heat transfer rate is 10 calories per second to maintain temperature of the patient, then the target heat transfer rate of the heat source can be 12 calories per second.

In block 512, the controller determines heat settings for the heat source. The heat settings cause the heat source to produce a certain heat transfer rate. The heat settings can factor in blower speed, temperature of air flow, cycle time, or combinations thereof.

In block 514, the controller can determine whether heat settings will produce the heat transfer rate. If the heat transfer rate may be unachievable due to resource constraints (such as battery life), then the controller can activate a secondary heating circuit in block 518. The secondary heating circuit can make up thermal deficiencies in the heat source. For example, if a convective heat source is designed for comfort warming, then the heat source may not have enough thermal power to warm the patient device. The secondary heating circuit can be used to increase the heat transfer rate of the convective heat source.

If the heat settings will produce the heat transfer rate, then the method 500 can continue to block 516 where the heat source is directed by the controller to produce heat according to the heat settings.

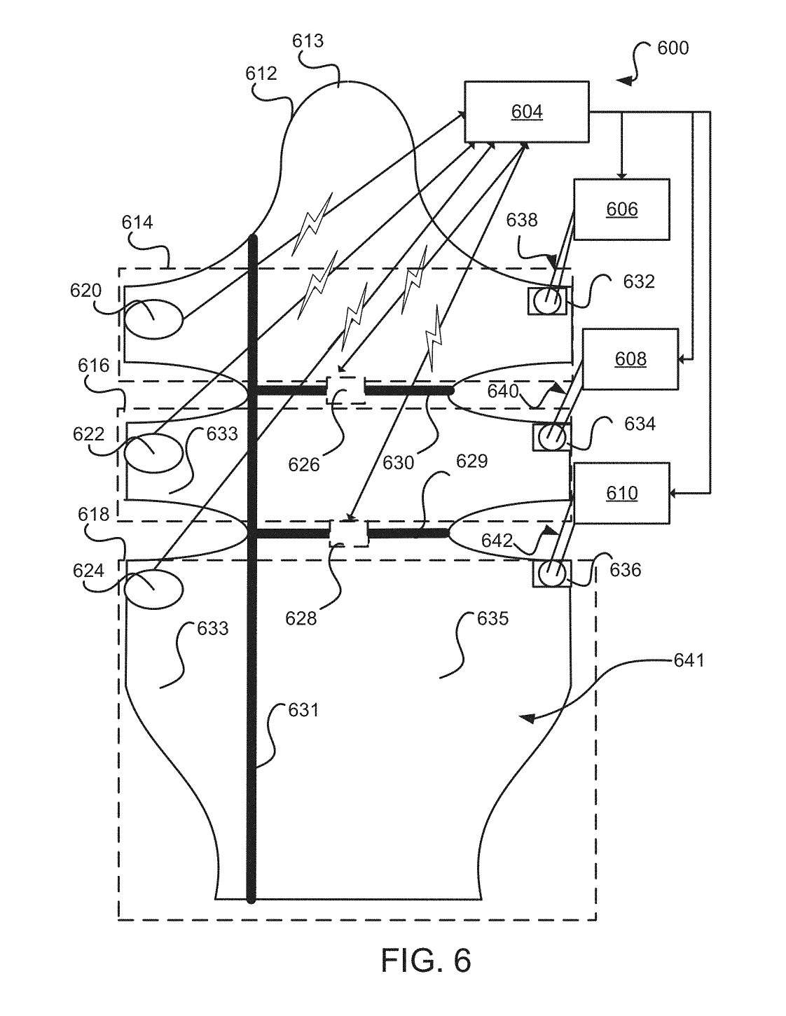

FIG. 6 illustrates a convective warming system (“system”) 600 utilizing some of the aspects disclosed herein. In this particular embodiment, the system utilizes a forced air warming blanket 612 (“blanket”) which is a type of warming device. The system 600 can have a controller 604 that is communicatively coupled to a plurality of convective heat sources (606, 608, 610) (“warming units”).

The controller 604 is communicatively coupled to the warming units 606, 608, 610, the valves 626, 628, and the temperature sensors 620, 622, 624. In this example, the controller 604 can be wired to the warming units 606, 608, 610, and form wireless communication with valves 626, 628, and the temperature sensors 620, 622, 624. In at least one embodiment, the controller 604 can wirelessly communicate with the warmings units 606, 608, 610.

In at least one embodiment, each warming unit 606, 608, 610 is a comfort warming unit which generally has a lower power output than other warming units such as a clinical warming unit. Each warming unit can be coupled to a hose portion (described herein). For example, warming unit 606 is coupled to hose portion 638 which is releasably attached to inlet port 632, warming unit 608 is coupled to hose portion 640 which is releasably attached to inlet port 634, warming unit 610 is coupled to hose portion 642 which is releasably attached to inlet port 636.

The inlet ports can provide a fluidic link between the warming units and internal air passageways of the blanket 612. The blanket 612 can have a dual layer construction. For example, the internal air passageways (also referred to as an interior space of the blanket 612) are formed from a first sheet of material 641 (which may be air-permeable) and a second sheet of material (not shown) of material bonded together adjacent a periphery using a peripheral seal 613 which may be formed through ultrasonic or heat bonding.

In at least one embodiment, a plurality of linear seals can define multiple zones within the blanket 612. For example, the blanket 612 can be divided into a plurality of zones through multiple linear seals 629, 630. The linear seals 629, 630 function to fluidically isolate the plurality of zones. For example, the first zone 614 can be separated from the blanket 612 by linear seals 630 and the valve 626. In at least one embodiment, a linear seal 630 can be positioned about a valve 626 such that the only way that air can pass between the first zone 614 and second zone 616 is through the valve 626. For example, the medial portions of the linear seal 630 pass between a valve 626. Likewise, linear seals 629 and 630 both fluidically isolate zone 616. Linear seal 629 fluidically isolates zone 618. In at least one embodiment, the valves 626, 628 are electromechanical and are configured to control the passage of air and maintain an airtight seal with the linear seals 629, 630.

In at least one embodiment, the blanket 612 has a plurality of temperature sensors disposed on any surface of the first sheet 641, the second sheet, or combinations thereof. In at least one embodiment, the plurality of temperature sensors 620, 622, 624 can be thermally separated from the heat applicator. In the blanket 612, this may be accomplished by at least one linear seal 631. For example, the linear seal 631 can contact at least one point of the peripheral seal 613 to form a non-inflatable area 633. In this example, the plurality of temperature sensors 620, 622, 624 can be thermally separated from the inflatable area 635 of the blanket (i.e., a heat applicator described herein).

In operation, the blanket 612 can wrap around a patient. The plurality of temperature sensors 620, 622, 624 can wirelessly provide sensor readings to the controller 604 which can control the heat transfer rate of the zones 614, 616, 618 within the inflatable area 635 through both the generation of heat by the plurality of warming units 606, 608, 610 and the interzone/intra blanket transfer facilitated by valves 626, 628.

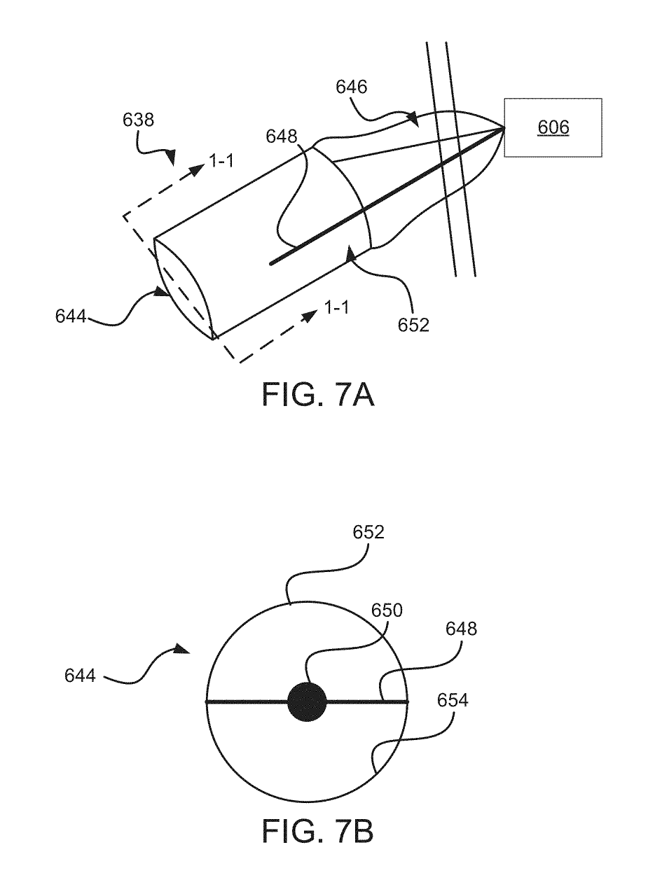

FIGS. 7A-B illustrate a hose portion 638. The hose portion 638 can be configured to deliver a stream of pressurized air into a blanket 612. The hose portion 638 can include a hose 646. The hose 646 can be generally flexible. One or more temperature sensors or air flow sensors may also be present within the hose 646. The hose 646 can couple to the warming unit 606.

The hose portion 638 can include a nozzle tip 644 that is configured to mate with an inlet port 632. The nozzle tip 644 can have an outer surface 652 which is configured to mate with an inlet port 632. The nozzle tip 644 can also have an inner surface 654.

In at least one embodiment, a wire 648 may be threaded through the nozzle tip 644 on both the inner surface 654 and the outer surface 652. The wire 648 may also be threaded through the resistive element 650. The wire 648 can be further coupled to a power source. The power source may be present in the warming unit 606. In at least one embodiment, an electrical current can flow through the wire 648 and through the resistive element 650 causing the resistive element 650 to emit heat. In at least one embodiment, the resistive element 650 can form a secondary heating circuit sufficient to heat air that flows through the hose portion 638.

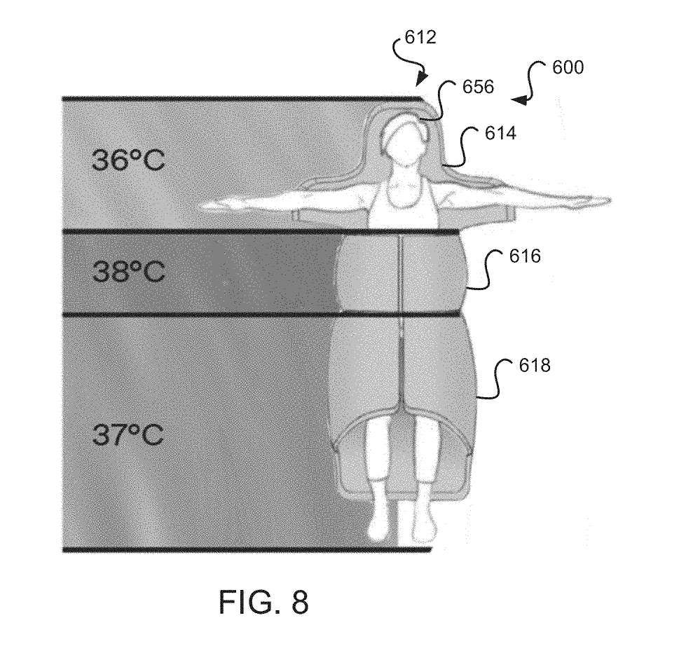

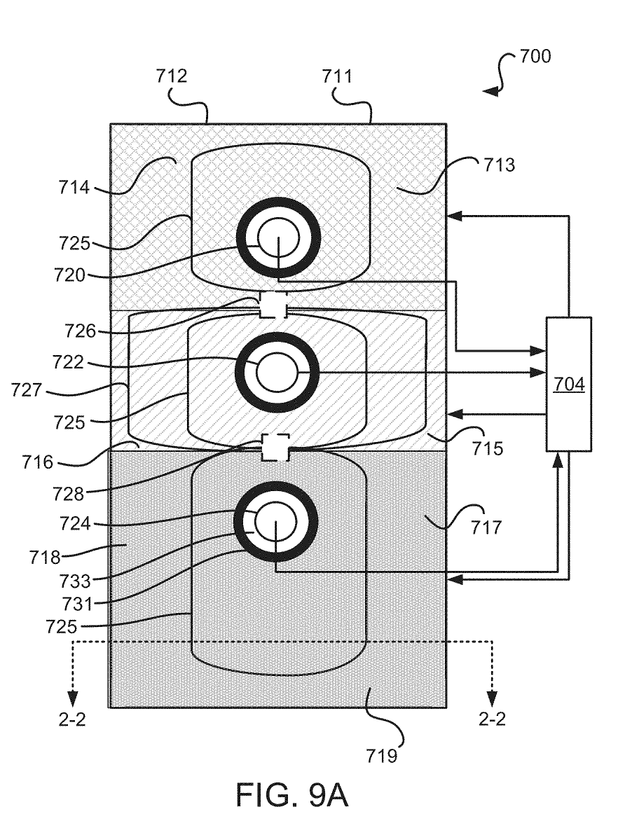

FIG. 8 illustrates the blanket 600 folded over the medial plane of the patient 656 such that the patient 656 is swaddled by the blanket 612. A plurality of zones 614, 616, 618 corresponding to portions of a body of the patient 656. Each zone has a different thermal profile. For example, zone 614 corresponds to the upper torso of the patient 656 with a sensor reading of 36 degrees C. Zone 616 corresponds to the abdominal cavity and core of the patient 656 with a sensor reading of 38 degrees C. Zone 618 corresponds to the leg extremities of the patient 656 with a sensor reading of 37 degrees C.

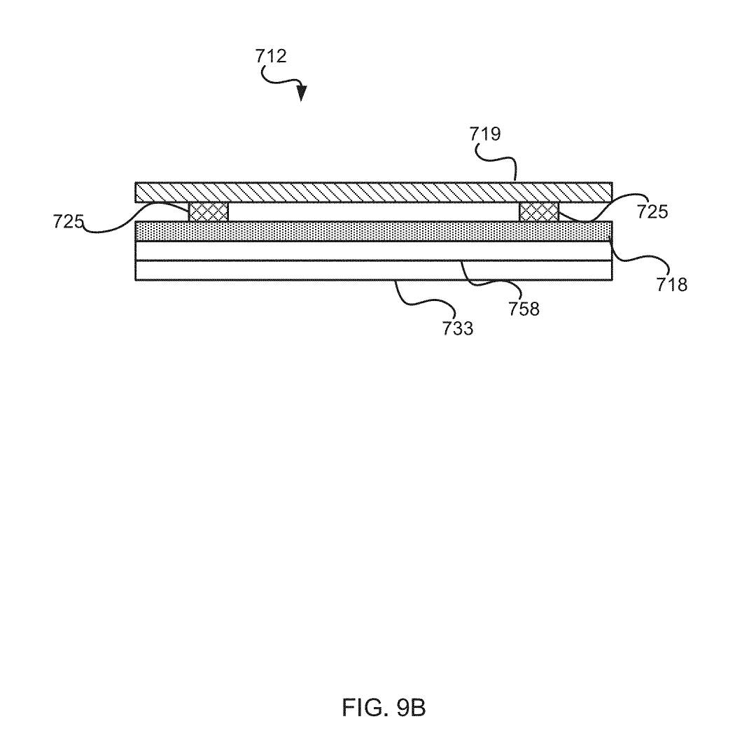

FIG. 9A illustrates a conductive heating system 700 (“system”). The system 700 can operate based on a plurality of conductive heat sources 714, 716, 718 (“resistive elements”). The conductive heating system 700 includes a warming unit 712 having a periphery 711.

Within the boundaries established by the periphery 711, the warming unit 712 can have a plurality of resistive elements 714, 716, 718 disposed therein. The resistive elements can define boundaries of a plurality of zones 713, 715, 717. For example, resistive element 714 defines a first zone 713, resistive element 716 defines a second zone 715, and resistive element 718 defines a third zone 717.

Each resistive element can have a cover layer 719 disposed thereon. Each resistive element can be electrically coupled to a power source (not shown) and communicatively coupled to the controller 704. Current flowing through the resistive element can produce heat. The controller 704 can be communicatively coupled to a plurality of sensors 720, 722, 724. Each sensor can transmit a sensor reading to the controller 704. The controller 704 can function the same as the controllers discussed herein.

Each sensor may also be thermally separated from the resistive element. In at least one embodiment, the thermal separation can include a sensor disposed on a patient comfort layer 733. The patient comfort layer 733 can be a material that is generally breathable such as cotton or a non-woven. The patient comfort layer can be removable to facilitate hygienic practices. The patient comfort layer 733 may be attached to the heating pad. The sensor may also be positioned to have a particular standoff distance from an edge 731 of the heating pad.

In at least one embodiment, the heat transfer element 726, 728 is a fluidic pump configured to pump a heat conductive liquid. The heat conductive liquid can transfer heat from one zone to another zone. The warming unit 712 can have one or more internal fluid channels 725, 727 fluidically coupled to the heat transfer elements 726, 728 and disposed proximate to the resistive elements 714, 716, 718. The heat conductive liquid can be stored within the internal fluid channels 725, 727.

Each heat transfer element can transfer heat from one zone to at least an adjacent zone. For example, heat transfer element 726 can transfer heat from zone 713 to zone 715 using fluid channel 725. Heat transfer element 728 can transfer heat from zone 715 to 717 using fluid channel 725. In at least one embodiment, a closed-loop system can be arranged to transfer heat from one zone to a proximate zone. For example, heat transfer element 726 can transfer heat from fluid channel 725 in zone 713 through fluid channel 727 along the periphery 711 and into zone 718 through 725. Thus, heat can be transferred from zone 713 to 717.

Turning to FIG. 9B, the fluid channels 725 can be disposed on the resistive element layer 718 (i.e., a plurality of heat sources). In particular, the fluid channels 725 can be positioned between the resistive element layer 718 and the cover layer 719. The cover layer 719 may function to protect the fluid channel 725 and resistive element 718. The cover layer 719 may also be reflective to help trap heat toward the patient. Disposed on another side of the resistive element layer 718 can be a layer 758. In at least one embodiment, the layer 758 can have a patient facing surface and an upper surface. The resistive element layer 718 can be disposed on the upper surface while the patient facing surface can have a patient comfort layer 733. In at least one embodiment, the layer 758 can be a heat applicator that disperses the heat from the resistive element layer 718. The heat applicator is described further herein. Disposed on the layer 758 is the patient comfort layer 733. In at least one embodiment, the resistive element comprises a layer of electrically conductive ink.

### LIST OF ILLUSTRATIVE EMBODIMENTS

1. A system comprising:

a warming device having at least a first zone and a second zone comprising:
a sensor,
a controller comprising:
one or more processor circuits configured to:
   receive a first sensor reading from the sensor corresponding to a first zone, wherein the first sensor reading corresponds to a first heat transfer rate;
   determine whether the sensor reading is sufficient;
   increase a first heat transfer rate to a second heat transfer rate in the first zone in response to the sensor reading being insufficient.
      2. The system of Embodiment 1, wherein the controller comprises one or more processor circuits configured to:

determine whether an excessive heat threshold is met responsive to the sensor reading being insufficient;

decrease the first heat transfer rate to a third heat transfer rate in the first zone in response to the excessive heat threshold being met.

3. The system of Embodiment 2, wherein the controller comprises one or more processor circuits configured to:

receive a second sensor reading in response to the excessive heat threshold not being met.

4. The system of any of Embodiments 1 to 3, further comprising: a heat transfer element configured to transfer heat between the first zone and the second zone.
5. The system of Embodiment 2, wherein the controller comprises one or more processor circuits configured to decrease the first heat transfer rate by transferring heat to a second zone using the heat transfer element.
6. The system of Embodiment 2, further comprising a heat source, wherein the controller comprises one or more processor circuits configured to decrease the first heat transfer rate by reducing heat received from the heat source.
7. The system of any of Embodiments 1 to 6, wherein the controller comprises one or more processor circuits configured to increase a first heat transfer rate in the first zone by:

increasing a fourth heat transfer rate to a fifth heat transfer rate from the second zone to the first zone, wherein the fourth heat transfer rate is negligible.

8. The system of Embodiment 7, wherein increasing the fourth heat transfer rate from the second zone comprises:

receiving a third sensor reading from a sensor corresponding to a second zone;

determining a second zone heat transfer rate present in the second zone;
determining whether the excessive heat threshold is met by the second zone heat transfer rate;
increasing a fourth heat transfer rate to a fifth heat transfer rate from the second zone to the first zone responsive to the excessive heat threshold being met.
9. The system of Embodiment 8, wherein the controller comprises one or more processor circuits configured to increase the first heat transfer rate in the first zone by transferring heat from a third zone in response to the excessive heat threshold not being met by the second zone heat transfer rate.
10. The system of Embodiment 9, wherein transferring heat from the third zone comprises:

receiving a fourth sensor reading from a sensor corresponding to a third zone;

determining a third zone heat transfer rate present in the third zone;
determining whether the excessive heat threshold is met by the third zone heat transfer rate;
transferring heat from the third zone responsive to the excessive heat threshold being met.
11. The system of any of Embodiments 1 to 10, wherein the controller comprises one or more processor circuits configured to increase the first heat transfer rate in the first zone by:

increasing a heat transfer rate in the heat source.

12. The system of Embodiment 11, wherein increasing a heat transfer rate in the heat source occurs in response to the excessive heat threshold not being met by the second zone heat transfer rate.
13. The system of Embodiment 11, wherein increasing a heat transfer rate in the heat source comprises:

determining a second heat transfer rate for the first zone;

determining heat settings of the heat source to produce the second heat transfer rate;

directing production of heat according to the heat settings.

14. The system of Embodiment 13, wherein increasing a heat transfer rate in the heat source comprises:

determining that the heat settings of the heat source will not produce the second heat transfer rate;

activating a secondary heating circuit based on the heat source not producing the second heat transfer rate.

15. The system of any of the preceding Embodiments, wherein the warming device is a convective warming device and the heat source is a heated air source.
16. The system of any of the preceding Embodiments, wherein the heat source comprises a hose and a hose nozzle.
17. The system of any of the preceding Embodiments, wherein the hose nozzle has an inner surface and an outer surface, wherein the secondary heating circuit is a resistive element disposed adjacent to the inner surface of the hose nozzle.
18. The system of any of the preceding Embodiments, wherein the warming device is a convective warming blanket.
19. The system of any of the preceding Embodiments, wherein the warming device is a convective warming gown.
20. The system of any of the preceding Embodiments, wherein the heat transfer element is an electromechanical valve communicatively coupled to the controller.
21. The system of any of the preceding Embodiments, wherein the heat applicator is an air permeable layer of material.
22. The system of any of the preceding Embodiments, wherein the warming device is a conductive warming device and the heat source is an electrical current.
23. The system of any of the preceding Embodiments, wherein the heat applicator is a resistive element.
24. The system of any of the preceding Embodiments, wherein the heat transfer element is an electromechanical valve coupled to a heat conductive liquid.
25. The system of any of the preceding Embodiments, wherein the secondary heating circuit comprises additional resistive elements separately controllable from the resistive element.
26. The system of any of the preceding Embodiments, wherein the controller comprises a communications module to communicate with the heat transfer element, the sensor, and the heat source.
27. The system of any of the preceding Embodiments, wherein the sensor is thermally isolated from the heat applicator.
28. The system of any of the preceding Embodiments, wherein transferring heat refers to increasing a heat transfer rate.
29. The system of any of the preceding Embodiments, wherein the one or more processor circuits are configured to determine whether the sensor reading is sufficient by:

determining the first heat transfer rate of the first zone; a

determining a first zone threshold based on whether the first heat transfer rate will cause the patient to maintain a range of temperatures;

determining whether the first heat transfer rate is within the first zone threshold.

30. The system of any of the preceding Embodiments, further comprising a patient.
31. The system of Embodiment 30, wherein the sensor reading is insufficient based on whether the first heat transfer rate will cause the patient to reduce body temperature below normal body temperature.
32. The system of Embodiment 30, wherein the sensor measures physiological parameters of the patient.
33. The system of any of Embodiments 30 to 32, wherein a zone corresponds to an extremity of the patient.
34. The system of any of Embodiments 30 to 33, wherein a zone corresponds to the core of the patient.
35. The system of any of the preceding Embodiments, wherein the warming device comprises a heat applicator.
36. A conductive warming device comprising:

a layer having a patient facing surface and an upper surface;

a plurality of heat sources disposed on the upper surface and arranged to form a plurality of zones;

a plurality of sensors, at least one sensor is disposed in a zone from the plurality of zones, wherein the at least one sensor is thermally isolated from a heat source from a plurality of heat sources.

37. The conductive warming device of Embodiment 36, comprising:

a heat transfer element arranged to transfer heat between two adjacent zones.

38. The conductive warming device of Embodiment 37, further comprising fluid channels fluidically coupled to the heat transfer element and disposed proximate to the plurality of heat sources.
39. The conductive warming device of Embodiment 38, wherein the fluid channels are configured to store heat conductive liquid in a closed-loop.
40. The conductive warming device of any of Embodiments 36 to Embodiment 39, wherein a heat source comprises a resistive element coupled to a power source.
41. The conductive warming device of Embodiment 40, wherein the resistive element comprises a layer of electrically conductive ink.
42. A system comprising:

the conductive warming device of any of Embodiment 36 to Embodiment 41;

a controller comprising one or more processor circuits and configured to:
receive a first sensor reading from a sensor corresponding to a first zone from the plurality of zones, wherein the first sensor reading corresponds to a first heat transfer rate;
determine whether the sensor reading is sufficient;
increase a first heat transfer rate to a second heat transfer rate in the first zone in response to the sensor reading being insufficient.
   43. The system of Embodiment 42, wherein the controller comprises one or more processor circuits configured to:

determine whether an excessive heat threshold is met responsive to the sensor reading being insufficient;

decrease the first heat transfer rate to a third heat transfer rate in the first zone in response to the excessive heat threshold being met.

44. The system of Embodiment 43, wherein the controller comprises one or more processor circuits configured to:

receive a second sensor reading in response to the excessive heat threshold not being met.

45. The system of Embodiment 43, wherein the controller comprises one or more processor circuits configured to decrease the first heat transfer rate by transferring heat to a second zone using the heat transfer element.
46. The system of Embodiment 43, wherein the controller comprises one or more processor circuits configured to decrease the first heat transfer rate by reducing heat received from the heat source.
47. The system of any of Embodiments 42 to 46, wherein the controller comprises one or more processor circuits configured to increase a first heat transfer rate in the first zone by:

increasing a fourth heat transfer rate to a fifth heat transfer rate from the second zone to the first zone, wherein the fourth heat transfer rate is negligible.

48. The system of Embodiment 47, wherein increasing the fourth heat transfer rate from the second zone comprises:

receiving a third sensor reading from a sensor corresponding to a second zone;

determining a second zone heat transfer rate present in the second zone;
determining whether the excessive heat threshold is met by the second zone heat transfer rate;
increasing a fourth heat transfer rate to a fifth heat transfer rate from the second zone to the first zone responsive to the excessive heat threshold being met.
49. A convective warming device comprising:

a first sheet of material and a second sheet of material bonded together at a peripheral seal, having an interior space formed therein;

an inlet port configured to couple with a heat source;

a plurality of zones and at least one non-inflatable area formed from one or more linear seals bonding portions of the first sheet and second sheet within the peripheral seal, wherein the plurality of zones are fluidically coupled to the inlet port; and

a plurality of sensors disposed within the non-inflatable area on the first sheet or the second sheet.

50. The convective warming device of Embodiment 48, wherein each zone has a sensor and an inlet port.
51. The convective warming device of Embodiment 48 or 49, wherein the convective warming device is configured to swaddle a patient.
52. The convective warming device of any of Embodiments 48 to 50, further comprising one or more heat transfer elements configured to transfer heat between the plurality of zones.
53. A system comprising:

the convective warming device of any of Embodiments 48 to 51;

a comfort warming unit.

54. The system of Embodiment 52, further comprising: a clinical warming unit.
55. The system of Embodiment 52, further comprising: a second comfort warming unit.
56. The system of Embodiment 52, further comprising:

a controller comprising one or more processor circuits and configured to:
receive a first sensor reading from a sensor corresponding to a first zone from the plurality of zones, wherein the first sensor reading corresponds to a first heat transfer rate;
determine whether the sensor reading is sufficient;
increase a first heat transfer rate to a second heat transfer rate in the first zone in response to the sensor reading being insufficient.
   57. The system of Embodiment 56, wherein a first comfort warming unit is fluidically coupled to the first zone and a second comfort warming unit is configured to a second zone, wherein the first zone is fluidically coupled to the second zone by a heat transfer element, wherein the one or more processor circuits are configured to increase the first heat transfer rate by

increasing a heat transfer rate of the second comfort warming unit and activating the heat transfer element.

58. A method comprising:

receiving a first sensor reading from a sensor corresponding to a first zone, wherein the first sensor reading corresponds to a first heat transfer rate;

determining whether the sensor reading is sufficient;

increasing a first heat transfer rate to a second heat transfer rate in the first zone in response to the sensor reading being insufficient.

59. The method of Embodiment 58, further comprising:

determining whether an excessive heat threshold is met responsive to the sensor reading being insufficient;

decreasing the first heat transfer rate to a third heat transfer rate in the first zone in response to the excessive heat threshold being met.

60. The method of Embodiment 59, further comprising:

receiving a second sensor reading in response to the excessive heat threshold not being met.

61. The method of Embodiment 59, further comprising: decreasing the first heat transfer rate by transferring heat to a second zone using the heat transfer element.
62. The method of Embodiment 59, further comprising decreasing the first heat transfer rate by reducing heat received from the heat source.
63. The method of any of Embodiments 58 to 62, wherein increasing a first heat transfer rate in the first zone comprises:

increasing a fourth heat transfer rate to a fifth heat transfer rate from the second zone to the first zone, wherein the fourth heat transfer rate is negligible.

64. The method of Embodiment 63, wherein increasing the fourth heat transfer rate from the second zone comprises:

receiving a third sensor reading from a sensor corresponding to a second zone;

determining a second zone heat transfer rate present in the second zone;
determining whether the excessive heat threshold is met by the second zone heat transfer rate;
increasing a fourth heat transfer rate to a fifth heat transfer rate from the second zone to the first zone responsive to the excessive heat threshold being met.
65. The method of Embodiment 64, wherein increasing the first heat transfer rate in the first zone comprises transferring heat from a third zone in response to the excessive heat threshold not being met by the second zone heat transfer rate.
66. The method of Embodiment 65, wherein transferring heat from the third zone comprises:

receiving a fourth sensor reading from a sensor corresponding to a third zone;

determining a third zone heat transfer rate present in the third zone;
determining whether the excessive heat threshold is met by the third zone heat transfer rate;
transferring heat from the third zone responsive to the excessive heat threshold being met.
67. The method of any of Embodiments 58 to 66, wherein increasing the first heat transfer rate in the first zone comprises increasing a heat transfer rate in the heat source.
68. The method of Embodiment 67, wherein increasing a heat transfer rate in the heat source occurs in response to the excessive heat threshold not being met by the second zone heat transfer rate.
69. The method of Embodiment 67, wherein increasing a heat transfer rate in the heat source comprises:

determining a second heat transfer rate for the first zone;

determining heat settings of the heat source to produce the second heat transfer rate;

directing production of heat according to the heat settings.

70. The method of Embodiment 69, wherein increasing a heat transfer rate in the heat source comprises:

determining that the heat settings of the heat source will not produce the second heat transfer rate;

activating a secondary heating circuit based on the heat source not producing the second heat transfer rate.

What is claimed is:

1. A system comprising:
a warming device having at least a first zone and a second zone comprising:
a sensor,
a controller comprising:
one or more processor circuits configured to:
receive a first sensor reading from the sensor corresponding to a first zone, wherein the first sensor reading corresponds to a first heat transfer rate;
determine whether the sensor reading is sufficient;
increase a first heat transfer rate to a second heat transfer rate in the first zone in response to the sensor reading being insufficient.

2. The system of claim 1, wherein the controller comprises one or more processor circuits configured to:
determine whether an excessive heat threshold is met responsive to the sensor reading being insufficient;
decrease the first heat transfer rate to a third heat transfer rate in the first zone in response to the excessive heat threshold being met.

3. The system of claim 2, wherein the controller comprises one or more processor circuits configured to:
receive a second sensor reading in response to the excessive heat threshold not being met.

4. The system of claim 1, further comprising: a heat transfer element configured to transfer heat between the first zone and a second zone.

5. The system of claim 4, wherein the controller comprises one or more processor circuits configured to decrease the first heat transfer rate by transferring heat to the second zone using the heat transfer element.

6. The system of claim 2, further comprising a heat source, wherein the controller comprises one or more processor circuits configured to decrease the first heat transfer rate by reducing heat received from the heat source.

7. The system of claim 4, wherein the controller comprises one or more processor circuits configured to increase a first heat transfer rate in the first zone by:
increasing a fourth heat transfer rate to a fifth heat transfer rate from the second zone to the first zone, wherein the fourth heat transfer rate is negligible.

8. The system of claim 7, wherein increasing the fourth heat transfer rate from the second zone comprises:
receiving a third sensor reading from the sensor corresponding to a second zone;
determining a second zone heat transfer rate present in the second zone;
determining whether an excessive heat threshold is met by the second zone heat transfer rate;
increasing a fourth heat transfer rate to a fifth heat transfer rate from the second zone to the first zone responsive to the excessive heat threshold being met.

9. The system of claim 8, wherein the controller comprises one or more processor circuits configured to increase the first heat transfer rate in the first zone by transferring heat from a third zone in response to the excessive heat threshold not being met by the second zone heat transfer rate.

10. The system of claim 9, wherein transferring heat from the third zone comprises:
receiving a fourth sensor reading from a sensor corresponding to a third zone;
determining a third zone heat transfer rate present in the third zone;
determining whether the excessive heat threshold is met by the third zone heat transfer rate;
transferring heat from the third zone responsive to the excessive heat threshold being met.

11. The system of claim 4, wherein the controller comprises one or more processor circuits configured to increase the first heat transfer rate in the first zone by:
increasing a heat transfer rate in the heat source.

12. The system of claim 11, wherein increasing a heat transfer rate in the heat source occurs in response to an excessive heat threshold not being met by the second zone heat transfer rate.

13. The system of claim 11, wherein increasing a heat transfer rate in the heat source comprises:
determining a second heat transfer rate for the first zone;
determining heat settings of the heat source to produce the second heat transfer rate;
directing production of heat according to the heat settings.

14. The system of claim 13, wherein increasing a heat transfer rate in the heat source comprises:
determining that the heat settings of the heat source will not produce the second heat transfer rate;
activating a secondary heating circuit based on the heat source not producing the second heat transfer rate.

15. The system of claim 1, wherein the sensor is thermally isolated from a heat applicator.

16. The system of claim 1, wherein the one or more processor circuits are configured to determine whether the sensor reading is sufficient by:
determining the first heat transfer rate of the first zone;
determining a first zone threshold based on whether the first heat transfer rate will cause a patient to maintain a range of temperatures;
determining whether the first heat transfer rate is within the first zone threshold.

17. The system of claim 1, further comprising a patient.

18. The system of claim 17, wherein the sensor reading is insufficient based on whether the first heat transfer rate will cause the patient to reduce body temperature below normal body temperature.

19. The system of claim 17, wherein the sensor measures physiological parameters of the patient.

20. The system of claim 17, wherein a zone corresponds to an extremity of the patient.
